(19) United States
(12) Patent Application Publication
(10) Pub. No.: US 2017/0028160 A1
(43) Pub. Date: Feb. 2, 2017

(54) RESPIRATORY THERAPY DEVICES

(71) Applicant: SMITHS MEDICAL INTERNATIONAL LIMITED, Kent (GB)

(72) Inventor: Mark Charles OLIVER, Hertfordshire (GB)

(73) Assignee: SMITHS MEDICAL INTERNATIONAL LIMITED, Kent (GB)

(21) Appl. No.: 15/303,833

(22) PCT Filed: Mar. 23, 2015
(86) PCT No.: PCT/GB2015/000097

§ 371 Date: Oct. 13, 2016

(30) Foreign Application Priority Data
Apr. 30, 2014 (GB) 1407571.7

(51) Int. Cl. A61M 16/20; A61M 16/00

(52) U.S. Cl. CPC A61M 16/202; A61M 16/0006; A61M 2016/003; A61M 2016/0027; A61M 2205/52; A61M 2205/586

(57) ABSTRACT

An expiratory therapy device (100) has a disc (21) mounted at right angles in the expiratory gas passage (9). The disc (21) is rotated about an axis at right angles to the passage (9) by an electric motor (23) at a speed that is independent of the respiratory pressure exerted by the patient during use. The motor (23) and its control (30) are located in a drive unit (25) that can be removed from the device (100) for reuse on another device.

This invention relates to respiratory therapy devices of the kind arranged to produce an alternating resistance to respiratory flow through the device, the device including a gas passage, a member movable relative to the gas passage to obstruct or enable gas flow through the passage.

Positive expiratory pressure (PEP) devices, that is, devices that present a resistance to expiration through the device, are now widely used to help treat patients suffering from a range of respiratory impairments, such as chronic obstructive pulmonary disease, bronchitis, cystic fibrosis and atelectasis. More recently, such devices that provide an alternating resistance to flow have been found to be particularly effective. One example of such a device is sold under the trade mark Acapella (a registered trade mark of Smiths Medical) by Smiths Medical and is described in U.S. Pat. No. 6,581,598, U.S. Pat. No. 6,776,159, U.S. Pat. No. 7,059,324 and U.S. Pat. No. 7,699,054. U.S. Pat. No. 8,534,284 describes a device with an interrupter valve driven by pressurised gas delivered to the apparatus. The speed of the valve is dependent on the back pressure created by expired breaths from the patient. Other vibratory respiratory therapy devices are available, such as “Quake” manufactured by Thayer, “AeroPEP” manufactured by Monaghan, “TheraPEP” manufactured by Smiths Medical and “IPV Percussionator” manufactured by Percussionaire Corp. These devices generate vibratory positive pressures mechanically and fluctuating exhalation flows that help overcome the inertia and stiction of the sputum within the bronchi and lower passages of the lung. This enhances mucociliary clearance. Alternative apparatus such as “CoughAssist” manufactured by Philips is also available. Respiratory therapy devices can instead provide an alternating resistance to flow during inhalation.

One possible problem with such devices is that the frequency of vibration is dependent on the expiration force exerted by the patient and this is not necessarily the optimum frequency for most effective treatment.

Furthermore, the frequency of operation, the resistance and flow through previous devices cannot be easily altered independently. Also, the waveform of the back pressure and flow pulse applied to the patient cannot be altered. Furthermore, these settings are dependent on the flow produced by the patient. So, although these devices can be very effective, clinicians often are unsure of the most effective setting for the frequency of the vibratory pulse or the restriction required providing optimum therapy performance for that particular patient with their specific condition at that specific time. Also, because the vibratory frequency is often linked inextricably with the change of exhalation flow rates and the restriction provided by the device it is difficult to know which parameters are more important and those that can be compromised to provide the most effective therapy. A further problem is that users often neglect to use the devices correctly as recommended by the physician or do not record what settings they have used, when and for how long.

The effectiveness of treatments by such V-PEP devices is thought to be critically dependent on the frequency, pressure, amplitudes and shape of the generated vibration or pulses.

It is an object of the present invention to provide an alternative respiratory therapy device.

According to the present invention there is provided a respiratory therapy device of the above-specified kind, characterised in that the device includes an electrical actuator arranged to move the movable member between obstructing and enabling states, the frequency of movement between the two states being substantially independent of respiratory pressure exerted by the patient during use of the device.

The electric actuator is preferably arranged to rotate or angularly displace the movable member relative to the gas passage. The movable member may be rotatable about an axis at right angles to the gas passage. The electrical actuator preferably includes an electric motor. The device may include a gear connected between the motor and the movable member. The device may include a sensor responsive to flow or pressure in the gas passage. The device preferably includes a memory arranged to store information about use of the device. The electrical actuator is preferably contained in a drive unit, which may be removable from the device for reuse. The drive unit is preferably shaped to provide a handle for using the device.

A vibratory PEP device according to the present invention will now be described, by way of example, with reference to the accompanying drawings, in which:

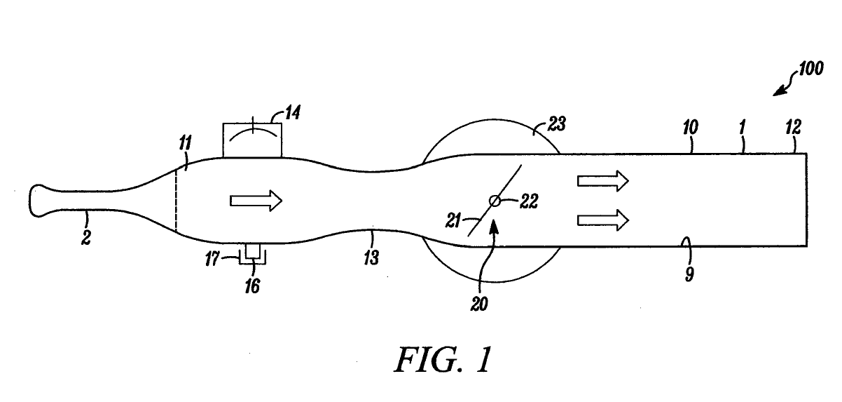

FIG. 1 is side elevation view of a first form of device;

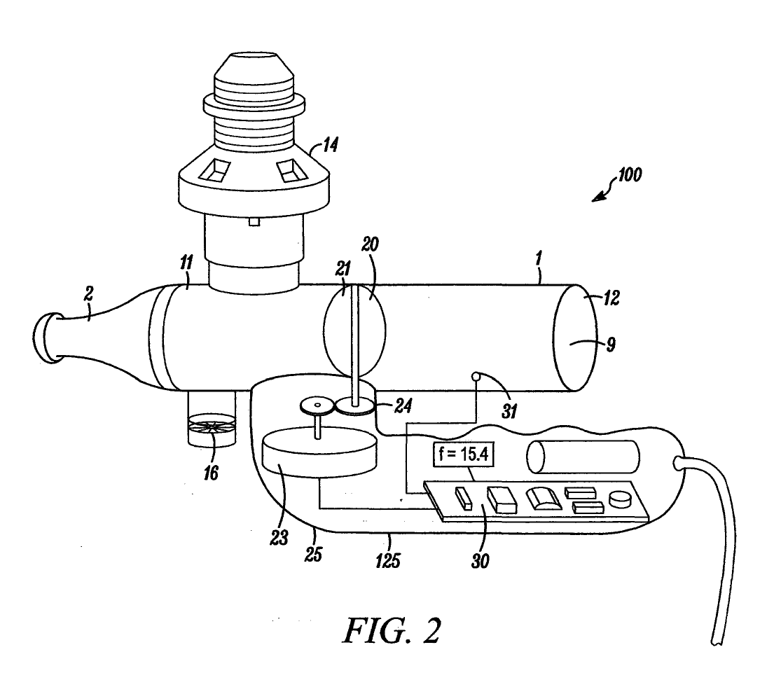

FIG. 2 is a perspective view of the device;

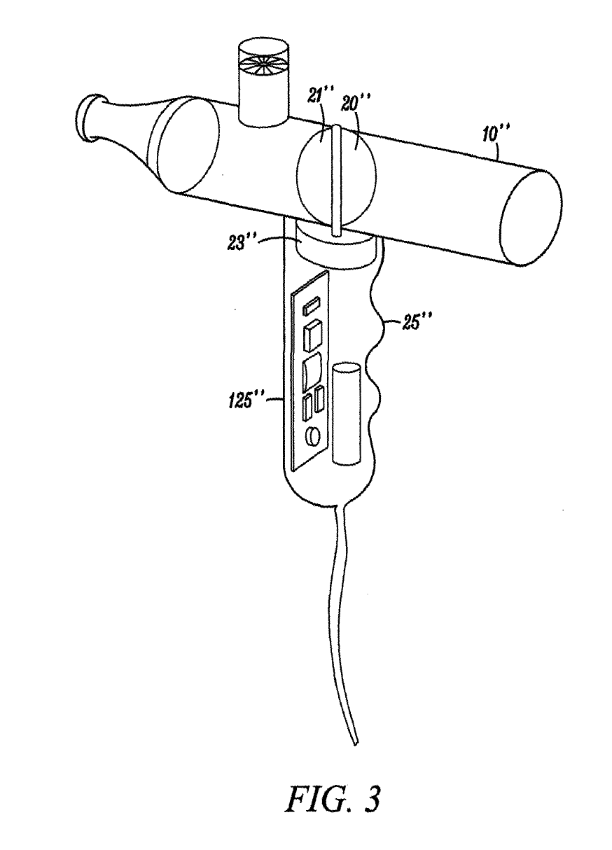

FIG. 3 is a perspective view of a modified form of the device; and

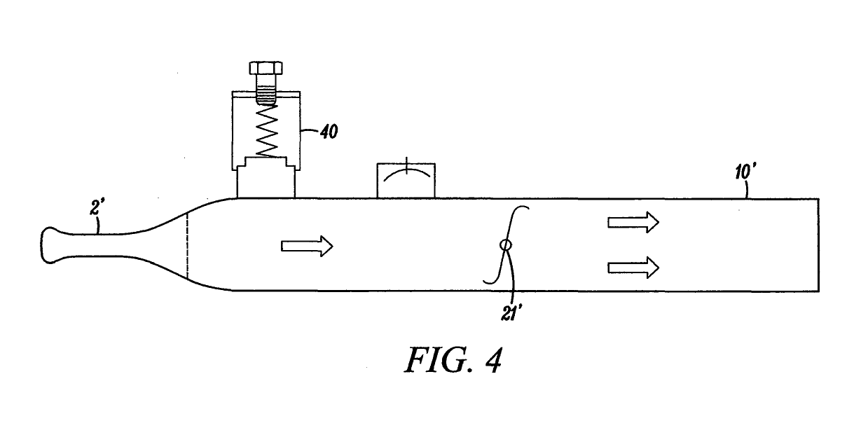

FIG. 4 is a side elevation of another modified form of device.

With reference first to FIGS. 1 and 2, the device 100 comprises a main body portion 1 and a separate, removable mouthpiece 2 fitted onto one end of the body portion. The body portion 1 includes an elongate tubular outer gas flow housing 10 with a gas passage 9 extending internally along it. The left-hand, inlet end 11 of the housing 10 is shaped to receive the mouthpiece 2 in mating connection and the opposite, outlet end 12 is open to air. The housing 10 has a substantially constant cross-section along its length and has an optional short section 13 about one third the way along the housing from its inlet end that is reduced in cross section to provide a slight constriction. Towards its inlet end 11 the housing 10 has a one-way valve 14 (shown schematically in FIG. 1) opening at opposite ends to atmosphere and to the interior of the housing respectively. The valve 14 is arranged to close and prevent flow through the valve when there is an elevated pressure within the housing 10 while the patient exhales. The valve 14 opens to allow air to flow into the housing 10 if there is a pressure drop within the housing caused by the patient inhaling, although normally the patient would remove the device from his mouth before inhaling. At the same location as the valve 14, on the underside of the housing 10, there is a drain port 16, normally closed by a cap 17 that can be removed to allow saliva to be drained away.

Adjustable means in the form of a restrictor valve 20 is mounted in the housing 10 just downstream of the optional constricted section 13. The valve 20 includes a movable member or means in the form of a flat, circular disc 21 mounted on an axle 22 extending diametrically in the plane of the disc and fixed orthogonally of the axis of the housing 10. The axle 22 is coupled with a rotary motor 23, such as a servo or stepper motor, or a piezo motor, via a gear arrangement 24 outside the gas-flow housing 10 in a drive unit 25 removably attached with the housing. The drive unit 25 is contained in a housing 125 extending parallel to and below the gas flow housing 10 and is shaped to form a handle or grip by which the device 100 can be held up to the mouth of the user. Rotation of the motor 23 rotates the gear 24 and causes the axle 22 to rotate about its axis, thereby causing the disc 21 to rotate between a position where it extends transversely at right angles to the axis of the housing 10 (an obstructing state), where it obstructs gas flow, providing the maximum restriction of the gas passage, to a position where plane of the disc is aligned with the axis of the housing (an enabling state), and the valve provides the minimum restriction, enabling gas flow along the passage through the housing 10.

The motor 23 is driven by a control unit 30 at a preprogramed speed to achieve the desired frequency, alternating between obstructing and enabling expiratory flow through the device 100, as set by the physician.

A flow or pressure sensor 31 is mounted inside the gas flow housing 10 to monitor flow or pressure in the housing. The output of the sensor 31 is supplied to the control unit 30 where it is recorded for subsequently monitoring use of the device. The output of the sensor 31 could also be used to trigger the start of recording. The control unit 30 includes a downloadable embedded memory component or removable storage device in which is stored information about the duration of use, settings, frequency of operation and the like so that this information can be analysed to determine the effectiveness of the device at different settings.

Preferably the housing 125 containing the drive unit 25 is removable from the gas flow housing 10 so that the housing and valve 20 can be disposed of and the drive unit reused on another gas flow housing.

The valve used to produce the alternating restriction to flow could take many different forms. For example, instead of being flat, the valve disc 21′ could have a curved shape as shown in FIG. 4. This arrangement also shows a PEEP valve 40 adjacent the mouthpiece 2′ that may be a mechanical or electrical device and is arranged to open to allow gas to flow out of the housing 10′ at a defined maximum PEEP pressure for the patient.

The valve could be rotated about an axis parallel with the axis of the housing in front of an opening to a gas flow passage along the housing so that it alternately covers and uncovers the opening. Alternative valves could take the form of flaps or the like that are flexed or otherwise displaced to provide an alternating restriction to flow. The flap could be magnetic and could be opened or closed by means of a solenoid. An adjustable limit stop with feedback could also be used with such a flap. It will be appreciated that the valve does not need completely to obstruct or enable gas flow along a gas passage but that it just needs to provide sufficient difference in flow between its obstructing and enabling states to have a therapeutic effect.

The apparatus could have proportional electronic valves to restrict the exhalation path. This would have the benefit of being able to alter the waveform of the pressure wave experienced by the patient without the need to alter the shape of the valve.

FIG. 3 shows an alternative arrangement for the device where the drive unit 25″ is contained in a housing 125″ extending at right angles from the gas flow housing 10″ and where the motor 23″ is directly coupled to the valve 20″ without the interposition of a gear train.

The device of the present invention can be arranged to provide a constant programmed frequency of operation that is independent of the patient's expiratory effort. This can improve the effectiveness of the device by ensuring that it always operates at the optimum frequency for effectiveness on a particular patient. Because the valve is electrically operated the device can be battery powered without the need for an external gas supply or the like, making it particularly suitable for routine daily use at the patient's home. The information collected in the device may be used to develop therapies for particular conditions and also to develop changing therapies depending on how successful initial and subsequent treatments are and how acute they maybe—altering any of the parameters to learn the best course of treatment. In time, with sufficient clinical experience and expertise, various programmes can be developed to treat specific conditions and tailored to suit specific individuals.

1-9. (canceled)

10. A respiratory therapy device arranged to produce an alternating resistance to respiratory flow through the device, the device including a gas passage, a member movable relative to the gas passage to obstruct or enable gas flow through the passage, characterised in that the device includes an electrical actuator arranged to move the movable member between obstructing and enabling states, the frequency of movement between the two states being substantially independent of respiratory pressure exerted by the patient during use of the device.

11. A respiratory therapy device according to claim 10, characterised in that the electrical actuator is arranged to rotate or angularly displace the movable member relative to the gas passage.

12. A respiratory therapy device according to claim 11, characterised in that the movable member is rotatable about an axis at right angles to the gas passage.

13. A respiratory therapy device according to claim 10, characterised in that the electrical actuator includes an electric motor.

14. A respiratory therapy device according to claim 13, characterised in that the device includes a gear connected between the motor and the movable member.

15. A respiratory therapy device according to claim 10, characterised in that the device includes a sensor responsive to gas flow or pressure in the gas passage.

16. A respiratory therapy device according to claim 10, characterised in that device includes a memory arranged to store information about use of the device.

17. A respiratory therapy device according to claim 10, characterised in that the electrical actuator is contained in a drive unit, and that the drive unit is removable from the device for reuse.

18. A respiratory therapy device according to claim 17, characterised in that the drive unit is shaped to provide a handle for using the device.
